(19) United States
(12) Patent Application Publication
(10) Pub. No.: US 2004/0071652 A1
(43) Pub. Date: Apr. 15, 2004

(54) Foam-like cosmetic hair composition comprising at least a aminoplast-ether skeleton thickening polymer

(76) Inventors: Christine Dupuis, Paris (FR); Patrick Minou, Taverny (FR)

Correspondence Address: FINNEGAN, HENDERSON, FARABOW, GARRETT & DUNNER, LLP, 1300 I STREET, NW, WASHINGTON, DC, 20005, US

(21) Appl. No.: 10/415,139

(22) PCT Filed: Oct. 25, 2001
(86) PCT No.: PCT/FR01/03317

(30) Foreign Application Priority Data
Oct. 27, 2000 (FR) 00/13867

(51) Int. Cl. A61K 7/06; A61K 7/11

(52) U.S. Cl. 424/70.17

(57) ABSTRACT

The invention concerns a foam-like cosmetic hair composition comprising, in a cosmetically acceptable medium, at least a thickening polymer with an aminoplast-ether skeleton. The invention also concerns its use for making a hair formulation such as hair care products, hair conditioning products or hair fixing and/or hair styling products.

The invention relates to a cosmetic hair composition in mousse form, comprising at least one thickening polymer with an aminoplast-ether skeleton.

The invention is also directed toward the use of said composition for the manufacture of hair formulations such as care products, conditioning products or products for fixing and/or holding the hair.

Cosmetic hair compositions generally contain at least one polymer, which is preferably anionic, nonionic or amphoteric. They may, for example, provide hair-fixing properties.

The formulations most commonly used are usually in the form of an aerosol mousse, an aerosol spray, a pump-bottle spray or a gel.

Mousses generally make it possible to obtain on the hair good distribution of the cosmetic compositions, and they are also easy to use. Since the polymers used in these products are generally nonfoaming or only sparingly foaming, it is necessary to add a foaming agent and/or an agent for improving the quality of the mousse.

The foaming agents and/or agents for improving the quality of the mousse that are generally used are, for example, anionic, nonionic or amphoteric surfactants. However, these surfactants occasionally give rise to refoaming on wet hair, which goes against a rapid and careful preparation of the hairstyle.

Moreover, the application of mousse to the hair occasionally has the drawback of making the hairstyle lank. Finally, either an aerosol device or a foam-pump device is required to produce mousses.

In general, the production of hair compositions in mousse form is achieved by means of introducing foaming surfactants or foaming polymers. In the first case, the composition often gives results that are relatively unsatisfactory in terms of cosmetic properties, and in the second case the foaming polymers risk being incompatible with the rest of the formulation. In addition, it is necessary to use a large amount of foaming polymer, which is prejudicial to the quality of the cosmetic results. These drawbacks moreover exist especially for leave-in foaming compositions.

In the specific case of foam pumps, the search for a compromise between the quality of the mousse and the cosmetic qualities is particularly difficult.

The problem posed by the invention is that of producing cosmetic compositions capable of foaming without involving a surfactant, and while using a limited amount of foaming agent, to obtain a mousse of satisfactory quality, and also satisfactory cosmetic properties.

The Applicant has found, surprisingly and unexpectedly, contrary to all expectation, that it is possible to produce cosmetic hair compositions in mousse form that do not have the drawbacks mentioned above, by selecting, as constituent of the cosmetic composition, a particular polymer that will be defined in greater detail hereinbelow.

One subject of the invention is a cosmetic hair composition in mousse form, comprising, in a cosmetically acceptable medium, at least one polymer with an aminoplast-ether skeleton.

For the purpose of the present invention, the term “aminoplast-ether” means any product derived from the condensation of an aldehyde with an amine or an amide.

For the purpose of the present invention, the term “aminoplast-ether” also means any structural unit formed from an aminoplast residue and a divalent hydrocarbon-based residue linked to the aminoplast residue via an ether bond.

Another subject of the invention relates to a cosmetic hair process using this composition.

Another subject of the invention relates more particularly to a process for fixing and/or holding the hair using this composition.

Yet another subject of the invention relates to the use of this composition for manufacturing hair products, especially those intended for fixing and/or shaping the hairstyle.

Yet another subject of the present invention relates to the use of a polymer with an aminoplast-ether skeleton, as a foaming agent in a cosmetic composition.

For the purpose of the present invention, the term “foam” means a microaerated structure of low density, which is well known to those skilled in the art.

The polymers with an aminoplast-ether skeleton that are used according to the invention are preferably chosen from those containing at least one unit of structure (I) below:

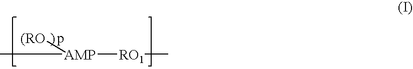

in which:

AMP is an aminoplast residue with alkylene units,

R denotes a hydrogen atom, a C₁-C₄ alkyl radical or a C₁-C₄ acyl radical,

RO₁ is a divalent alkyleneoxy residue,

p denotes a positive integer,

the group(s) OR being linked to the alkylene units of the AMP residue.

Preferably, the polymers with an aminoplast-ether skeleton are chosen from those containing at least one unit of structure (II) below:

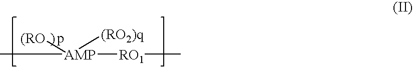

in which:

AMP, R, RO₁ and p have the same meaning as above,

RO₂ is a hydrophobic group other than RO linked to AMP via a hetero atom and comprising at least two carbon atoms, and

q is a positive integer.

Even more preferably, the polymers of the invention are of formulae (III) and (IIIa) below:

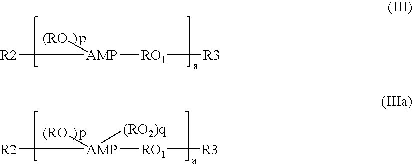

in which:

AMP, R, RO₁, RO₂, p and q have the same meaning as above, R2 or R3, which may be identical or different, represent an end group that can denote a hydrogen atom, a group RO₁H, a group RO₂H, a group AMP(OR)ₚ or any monofunctional group such as alkyl, cycloalkyl, aryl, aralkyl, alkylaryl, alkyloxyalkyl, aryloxyalkyl or cycloalkoxyalkyl,

a being a number greater than 1 and preferably greater than 2.

The aminoplast residues bearing the groups OR thereof integrated into the polymers of the invention may be chosen, in a nonlimiting manner, from structures (IV) to (XV) below:

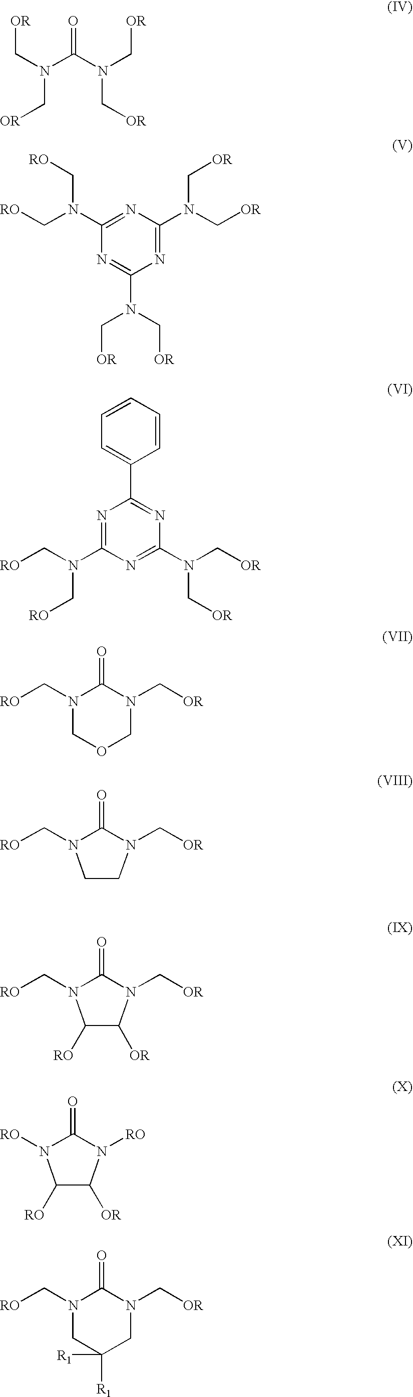

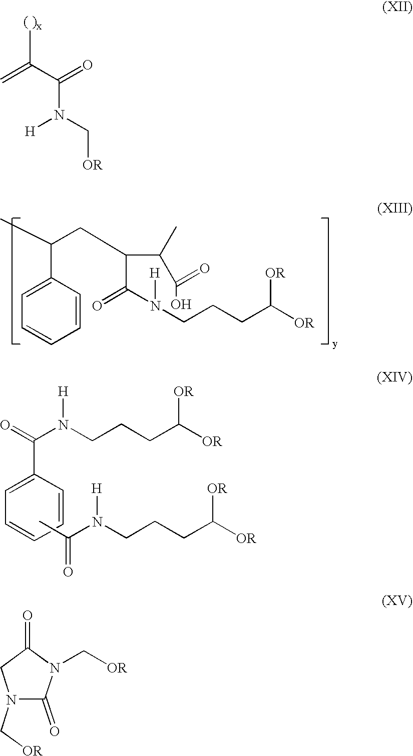

in which:

R has the same meaning as above,

R1 denotes C₁-C₄ alkyl,

y is a number at least equal to 2,

x denotes 0 or 1.

Preferably, the aminoplast residue(s) bearing the groups OR thereof is (are) chosen from those of structure (XVI) below:

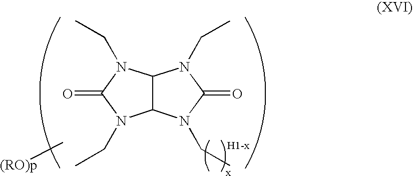

in which R, p and x have the same meanings as above.

The divalent alkyleneoxy residues are preferably those corresponding to the diols of general formula (XVII) below:

HO-(ZO)y-(Z1(Z2O)w)t-(Z′O)y′-Z3OH (XVII),

y and y′ being numbers ranging from 0 to 1000,

t and w being numbers ranging from 0 to 10,

Z, Z′, Z2 and Z3 are C₂-C₄ alkylene radicals and preferably radicals —CH2-CH(Z4)- and —CH2-CH(Z4)-CH2-,

Z1 being a linear or cyclic, branched or unbranched, aromatic or nonaromatic radical optionally comprising one or more hetero atoms and containing from 1 to 40 carbon atoms,

Z4 denoting a hydrogen atom or a C₁-C₄ alkyl radical or a C₁-C₃ acyl radical, it being understood that at least one of the radicals Z4 of the radicals Z, Z′, Z2 and Z3 is other than acyl.

Preferably, Z4 denotes a hydrogen atom or a methyl radical.

Even more preferably, t=0 and Z, Z′ and Z3 denote —CH2CH2-, and at least one of the groups from among y and y′ is other than 0. The compounds of formula (XVII) are then polyethylene glycols.

The aminoplast-ether polymers of formula (I) according to the invention are described in particular in patent U.S. Pat. No. 5,914,373, the content of which forms an integral part of the invention.

As polymers with an aminoplast-ether skeleton of formula (I), mention may be made in particular of the products Pure-Thix L [PEG-180/Octoxynol-40/TMMG Copolymer (INCI name)], Pure-Thix M [PEG-180/Laureth-50/TMMG Copolymer (INCI name)] and Pure-Thix HH [Polyether-1 (INCI name)] sold by the company Sud-Chemie, or Pure-Thix TX 1442, of tetrakis methoxy methyl glycoluril/hydrophobic modified polyether structure.

The polymers with an aminoplast-ether skeleton are preferably used in an amount that can range from about 0.05% to 3% by weight relative to the total weight of the composition. More preferably, this amount ranges from about 0.3% to 2% by weight.

The compositions in accordance with the invention may comprise a fixing polymer.

The cationic, anionic, amphoteric and nonionic fixing polymers that may be used in accordance with the invention are described below.

The cationic fixing polymers that may be used according to the present invention are preferably chosen from polymers comprising primary, secondary, tertiary and/or quaternary amine groups forming part of the polymer chain or directly attached thereto, and having a molecular weight of between 500 and about 5 000 000 and preferably between 1 000 and 3 000 000.

Among these polymers, mention may be made more particularly of the following cationic polymers:

(1) homopolymers or copolymers derived from acrylic or methacrylic esters or amides and comprising at least one of the units of the following formulae:

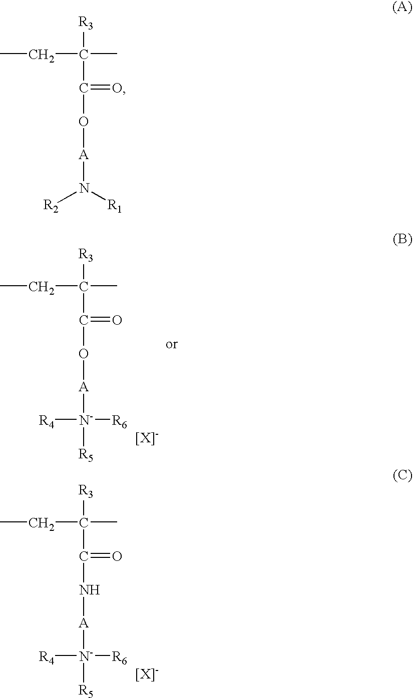

in which:

R₃ denotes a hydrogen atom or a CH₃ radical;

A is a linear or branched alkyl group of 1 to 6 carbon atoms or a hydroxyalkyl group of 1 to 4 carbon atoms;

R₄, R₅ and R₆, which may be identical or different, represent an alkyl group having from 1 to 18 carbon atoms or a benzyl radical;

R₁ and R₂ represent hydrogen or an alkyl group having from 1 to 6 carbon atoms;

X denotes a methosulfate anion or a halide such as chloride or bromide.

The copolymers of the family (1) also contain one or more comonomer units which can be chosen from the family of acrylamides, methacrylamides, diacetone acrylamides, acrylamides and methacrylamides substituted on the nitrogen with lower alkyls, acrylic or methacrylic acids or esters thereof, vinyllactams such as vinylpyrrolidone or vinylcaprolactam, and vinyl esters.

Thus, among these copolymers of the family (1), mention may be made of:

copolymers of acrylamide and of dimethylaminoethyl methacrylate quaternized with dimethyl sulfate or with a dimethyl halide, such as the one sold under the name Hercofloc by the company Hercules,

copolymers of acrylamide and of methacryloyloxyethyltrimethylammonium chloride, described, for example, in patent application EP-A-080 976 and sold under the name Bina Quat P 100 by the company Ciba Geigy,

the copolymer of acrylamide and of methacryloyloxyethyltrimethylammonium methosulfate, sold under the name Reten by the company Hercules,

quaternized or nonquaternized vinylpyrrolidone/dialkylaminoalkyl acrylate or methacrylate copolymers, such as the products sold under the name “Gafquat” by the company ISP, such as, for example, “Gafquat 734” or “Gafquat 755” or alternatively the products known as “Copolymer 845, 958 and 937”. These polymers are described in detail in French patents 2 077 143 and 2 393 573,

dimethylaminoethyl methacrylate/vinylcaprolactam/vinylpyrrolidone terpolymers, such as the product sold under the name Gaffix VC 713 by the company ISP, and

the quaternized vinylpyrrolidone/dimethyl-aminopropylmethacrylamide copolymer, such as the product sold under the name “Gafquat HS 100” by the company ISP;

(2) the quaternized polysaccharides described more particularly in American patents U.S. Pat. Nos. 3,589,578 and 4,031,307, such as guar gums containing trialkylammonium cationic groups;

Such products are sold in particular under the trade names Jaguar C13S, Jaguar C15 and Jaguar C17 by the company Meyhall.

(3) quaternary copolymers of vinylpyrrolidone and of vinylimidazole, such as the products sold by BASF under the name. Luviquat TFC;

(4) chitosans or salts thereof;

the salts that can be used are, in particular, chitosan acetate, lactate, glutamate, gluconate or pyrrolidonecarboxylate.

Among these compounds, mention may be made of chitosan having a degree of deacetylation of 90.5% by weight, sold under the name Kytan Brut Standard by the company Aber Technologies, and chitosan pyrrolidonecarboxylate sold under the name Kytamer PC by the company Amerchol.

(5) cationic cellulose derivatives such as copolymers of cellulose or of cellulose derivatives grafted with a water-soluble monomer comprising a quaternary ammonium, and disclosed in particular in patent U.S. Pat. No. 4,131,576, such as hydroxyalkylcelluloses, for instance hydroxymethyl-, hydroxyethyl- or hydroxypropylcelluloses grafted in particular with a methacryloyloxyethyltrimethylammonium, methacrylamidopropyltrimethylammonium or dimethyldiallylammonium salt.

The products sold corresponding to this definition are, more particularly, the products sold under the name “Celquat L 200” and “Celquat H 100” by the company National Starch.

The anionic fixing polymers generally used are polymers comprising groups derived from carboxylic acid, sulfonic acid or phosphoric acid and have a molecular weight of approximately between 500 and 5 000 000.

1) The carboxylic groups are provided by unsaturated monocarboxylic or dicarboxylic acid monomers such as those corresponding to the formula:

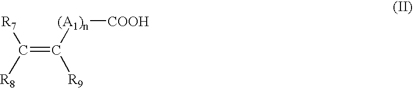

in which n is an integer from 0 to 10, A₁ denotes a methylene group, optionally connected to the carbon atom of the unsaturated group, or to the neighboring methylene group when n is greater than 1, via a hetero atom such as oxygen or sulfur, R₇ denotes a hydrogen atom or a phenyl or benzyl group, R₈ denotes a hydrogen atom or a lower alkyl or carboxyl group, R₉ denotes a hydrogen atom, a lower alkyl group or a —CH₂—COOH, phenyl or benzyl group.

In the abovementioned formula, a lower alkyl radical preferably denotes a group having 1 to 4 carbon atoms and in particular methyl and ethyl.

The anionic fixing polymers containing carboxylic groups that are preferred according to the invention are:

A) acrylic or methacrylic acid homo- or copolymers, or salts thereof and in particular the products sold under the names Versicol E or K by the company Allied Colloid and Ultrahold by the company BASF. Copolymers of acrylic acid and of acrylamide sold in the form of their sodium salt under the names Reten 421, 423 or 425 by the company Hercules, the sodium salts of polyhydroxycarboxylic acids.

B) copolymers of acrylic or methacrylic acids with a monoethylenic monomer such as ethylene, styrene, vinyl esters, acrylic or methacrylic acid esters, optionally grafted onto a polyalkylene glycol such as polyethylene glycol and optionally crosslinked. Such polymers are described in particular in French patent 1 222 944 and German patent application 2 330 956, the copolymers of this type comprising an optionally N-alkylated and/or hydroxyalkylated acrylamide unit in their chain as described in particular in Luxembourg patent applications 75370 and 75371 or sold under the name Quadramer by the company American Cyanamid. Mention may also be made of copolymers of acrylic acid and of C₁-C₄ alkyl methacrylate and terpolymers of vinylpyrrolidone, of acrylic acid and of C₁-C₂₀ alkyl methacrylate, for example of lauryl, such as the product sold by the company ISP under the name Acrylidone LM and methacrylic acid/ethyl acrylate/tert-butyl acrylate terpolymers such as the product sold under the name Luvimer 100 P by the company BASF.

C) copolymers derived from crotonic acid such as those comprising vinyl acetate or propionate units in their chain and optionally other monomers such as allylic esters or methallylic esters, vinyl ether or vinyl ester of a linear or branched saturated carboxylic acid with a long hydrocarbon chain such as those containing at least 5 carbon atoms, it being possible for these polymers optionally to be grafted and crosslinked, or alternatively a vinyl, allylic or methallylic ester of an α- or β-cyclic carboxylic acid. Such polymers are described, inter alia, in French patents 1 222 944, 1 580 545, 2 265 782, 2 265 781, 1 564 110 and 2 439 798. Commercial products falling into this class are the resins 28-29-30, 26-13-14 and 28-13-10 sold by the company National Starch.

D) copolymers derived from C₄-C₈ monounsaturated carboxylic acids or anhydrides chosen from:

copolymers comprising (i) one or more maleic, fumaric or itaconic acids or anhydrides and (ii) at least one monomer chosen from vinyl esters, vinyl ethers, vinyl halides, phenylvinyl derivatives, acrylic acid and its esters, the anhydride functions of these copolymers optionally being monoesterified or monoamidated. Such polymers are described in particular in US patents U.S. Pat. No. 2,047,398, U.S. Pat. No. 2,723,248 and U.S. Pat. No. 2,102,113 and GB patent GB 839 805 and in particular those sold under the names Gantrez AN or ES by the company ISP,

copolymers comprising (i) one or more maleic, citraconic or itaconic anhydrides and (ii) one or more monomers chosen from allylic or methallylic esters optionally comprising one or more acrylamide, methacrylamide, α-olefin, acrylic or methacrylic esters, acrylic or methacrylic acid or vinylpyrrolidone groups in their chain,

the anhydride functions of these copolymers optionally being monoesterified or monoamidated.

These polymers are described, for example, in French patents FR 2 350 384 and FR 2 357 241 by the Applicant.

E) polyacrylamides comprising carboxylate groups.

The polymers comprising sulfonic groups are polymers comprising vinylsulfonic, styrenesulfonic, naphthalenesulfonic or acrylamidoalkylsulfonic units.

These polymers can be chosen in particular from:

polyvinylsulfonic acid salts having a molecular weight of approximately between 1 000 and 100 000, as well as the copolymers with an unsaturated comonomer such as acrylic or methacrylic acids and their esters, as well as acrylamide or its derivatives, vinyl ethers and vinylpyrrolidone;

polystyrenesulfonic acid salts, the sodium salts having a molecular weight of about 500 000 and about 100 000, which are sold respectively under the names Flexan 500 and Flexan 130 by National Starch. These compounds are described in patent FR 2 198 719;

polyacrylamidesulfonic acid salts, those mentioned in patent U.S. Pat. No. 4,128,631 and more particularly polyacrylamidoethylpropanesulfonic acid sold under the name Cosmedia Polymer HSP 1180 by Henkel.

According to the invention, the anionic fixing polymers are preferably chosen from acrylic acid copolymers, such as the acrylic acid/ethyl acrylate/N-tert-butylacrylamide terpolymer sold under the name Ultrahold Strong by the company BASF, copolymers derived from crotonic acid, such as vinyl acetate/vinyl tert-butylbenzoate/crotonic acid terpolymers and the crotonic acid/vinyl acetate/vinyl neododecanoate terpolymers sold under the name Resin 28-29-30 by the company National Starch, polymers derived from maleic, fumaric or itaconic acids or anhydrides with vinyl esters, vinyl ethers, vinyl halides, phenylvinyl derivatives and acrylic acid and esters thereof, such as the methyl vinyl ether/monoesterified maleic anhydride copolymer sold under the name Gantrez ES 425 by the company ISP, the copolymers of methacrylic acid and of methyl methacrylate sold under the name Eudragit L by the company Rohm Pharma, the copolymer of methacrylic acid and of ethyl acrylate sold under the name Luvimer MAEX or MAE by the company BASF and the vinyl acetate/crotonic acid copolymer sold under the name Luviset CA 66 by the company BASF and the vinyl acetate/crotonic acid copolymer grafted with polyethylene glycol sold under the name Aristoflex A by the company BASF.

The anionic fixing polymers which are most particularly preferred are chosen from the methyl vinyl ether/monoesterified maleic anhydride copolymer sold under the name Gantrez ES 425 by the company ISP, the acrylic acid/ethyl acrylate/N-tert-butylacrylamide terpolymer sold under the name Ultrahold Strong by the company BASF, the copolymers of methacrylic acid and of methyl methacrylate sold under the name Eudragit L by the company Rohm Pharma, the vinyl acetate/vinyl tert-butylbenzoate/crotonic acid terpolymers and the crotonic acid/vinyl acetate/vinyl neododecanoate terpolymers sold under the name Resin 28-29-30 by the company National Starch, the copolymer of methacrylic acid and of ethyl acrylate sold under the name Luvimer MAEX or MAE by the company BASF and the vinylpyrrolidone/acrylic acid/lauryl methacrylate terpolymer sold under the name Acrylidone LM by the company ISP.

The amphoteric fixing polymers which can be used in accordance with the invention can be chosen from polymers comprising units B and C distributed randomly in the polymer chain, in which B denotes a unit derived from a monomer comprising at least one basic nitrogen atom and C denotes a unit derived from an acid monomer comprising one or more carboxylic or sulfonic groups, or alternatively B and C can denote groups derived from carboxybetaine or sulfobetaine zwitterionic monomers;

B and C can also denote a cationic polymer chain comprising primary, secondary, tertiary or quaternary amine groups, in which at least one of the amine groups bears a carboxylic or sulfonic group connected via a hydrocarbon radical or alternatively B and C form part of a chain of a polymer containing an α,β-dicarboxylic ethylene unit in which one of the carboxylic groups has been made to react with a polyamine comprising one or more primary or secondary amine groups.

The amphoteric fixing polymers corresponding to the definition given above which are more particularly preferred are chosen from the following polymers:

(1) polymers resulting from the copolymerization of a monomer derived from a vinyl compound bearing a carboxylic group such as, more particularly, acrylic acid, methacrylic acid, maleic acid, α-chloroacrylic acid, and a basic monomer derived from a substituted vinyl compound containing at least one basic atom, such as, more particularly, dialkylaminoalkyl methacrylate and acrylate, dialkylaminoalkylmethacrylamides and -acrylamides. Such compounds are described in patent U.S. Pat. No. 3,836,537.

(2) polymers comprising units derived from:

a) at least one monomer chosen from acrylamides and methacrylamides substituted on the nitrogen with an alkyl radical,

b) at least one acidic comonomer containing one or more reactive carboxylic groups, and

c) at least one basic comonomer such as esters containing primary, secondary, tertiary and quaternary amine substituents of acrylic and methacrylic acids and the product of quaternization of dimethylaminoethyl methacrylate with dimethyl or diethyl sulfate.

The N-substituted acrylamides or methacrylamides which are more particularly preferred according to the invention are groups in which the alkyl radicals contain from 2 to 12 carbon atoms and more particularly N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers are chosen more particularly from acrylic acid, methacrylic acid, crotonic acid, itaconic acid, maleic acid and fumaric acid and alkyl monoesters, having 1 to 4 carbon atoms, or maleic or fumaric acids or anhydrides.

The preferred basic comonomers are aminoethyl, butylaminoethyl, N,N′-dimethylaminoethyl and N-tert-butylaminoethyl methacrylates.

The copolymers whose CTFA (4th edition, 1991) name is octylacrylamide/acrylates/butylaminoethyl methacrylate copolymer, such as the products sold under the name Amphomer or Lovocryl 47 by the company National Starch, are particularly used.

(3) crosslinked and alkylated polyamino amides partially or totally derived from polyamino amides of general formula III:

[CO—R₁₀—CO-Z] (III)

in which R₁₀ represents a divalent radical derived from a saturated dicarboxylic acid, a mono- or dicarboxylic aliphatic acid containing an ethylenic double bond, an ester of a lower alkanol, having 1 to 6 carbon atoms, of these acids, or a radical derived from the addition of any one of said acids to a bis(primary) or bis(secondary) amine, and Z denotes a bis(primary), mono- or bis(secondary) polyalkylene-polyamine radical and preferably represents:

a) in proportions of from 60 to 100 mol %, the radical IV

—NH[(CH₂)ₓ—NH]ₚ (IV)

where x=2 and p=2 or 3, or alternatively x=3 and p=2

this radical being derived from diethylenetriamine, from triethylenetetraamine or from dipropylenetriamine;

b) in proportions of from 0 to 40 mol %, the radical (IV) above in which x=2 and p=1 and which is derived from ethylenediamine, or the radical derived from piperazine:

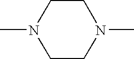

c) in proportions of from 0 to 20 mol %, the —NH—CH₂)₆—NH— radical derived from hexamethylenediamine, these polyamino amines being crosslinked by addition of a difunctional crosslinking agent chosen from epihalohydrins, diepoxides, dianhydrides and bisunsaturated derivatives, using from 0.025 to 0.35 mol of crosslinking agent per amine group of the polyamino amide and alkylated by the action of acrylic acid, chloroacetic acid or an alkane sultone, or salts thereof.

The saturated carboxylic acids are preferably chosen from acids having 6 to 10 carbon atoms, such as adipic acid, 2,2,4-trimethyladipic acid and 2,4,4-trimethyladipic acid, terephthalic acid, acids containing an ethylenic double bond such as, for example, acrylic acid, methacrylic acid and itaconic acid.

The alkane sultones used in the alkylation are preferably propane sultone or butane sultone, the salts of the alkylating agents are preferably the sodium or potassium salts.

(4) polymers comprising zwitterionic units of formula V:

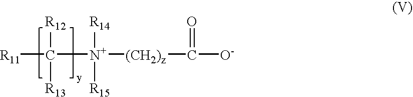

in which R₁₁ denotes a polymerizable unsaturated group such as an acrylate, methacrylate, acrylamide or methacrylamide group, y and z represent an integer from 1 to 3, R₁₂ and R₁₃ represent a hydrogen atom, methyl, ethyl or propyl, R₁₄ and R₁₅ represent a hydrogen atom or an alkyl radical such that the sum of the carbon atoms in R₁₄ and R₁₅ does not exceed 10.

The polymers comprising such units can also comprise units derived from nonzwitterionic monomers such as dimethyl or diethylaminoethyl acrylate or methacrylate or alkyl acrylates or methacrylates, acrylamides or methacrylamides or vinyl acetate.

By way of example, mention may be made of the copolymer of methyl methacrylate/methyl dimethylcarboxymethylammonioethylmethacrylate such as the product sold under the name Diaformer Z301 by the company Sandoz.

(5) polymers derived from chitosan comprising monomer units corresponding to the following formulae:

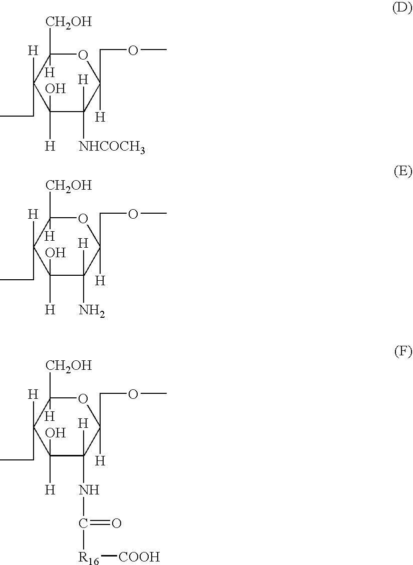

the unit D being present in proportions of between 0 and 30%, the unit E in proportions of between 5 and 50% and the unit F in proportions of between 30 and 90%, it being understood that, in this unit F, R₁₆ represents a radical of formula:

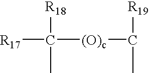

in which, if q=0, R₁₇, R₁₈ and R₁₉, which may be identical or different, each represent a hydrogen atom, a methyl, hydroxyl, acetoxy or amino residue, a monoalkylamine residue or a dialkylamine residue which are optionally interrupted by one or more nitrogen atoms and/or optionally substituted with one or more amine, hydroxyl, carboxyl, alkylthio or sulfonic groups, an alkylthio residue in which the alkyl group bears an amino residue, at least one of the radicals R₁₇, R₁₈ and R₁₉ being, in this case, a hydrogen atom;

or, if q=1, R₁₇, R₁₈ and R₁₉ each represent a hydrogen atom, as well as the salts formed by these compounds with bases or acids.

(6) polymers derived from the N-carboxyalkylation of chitosan, such as N-carboxymethylchitosan or N-carboxybutylchitosan sold under the name “Evalsan” by the company Jan Dekker.

(7) polymers corresponding to the general formula (VI) which are described, for example, in French patent 1 400 366:

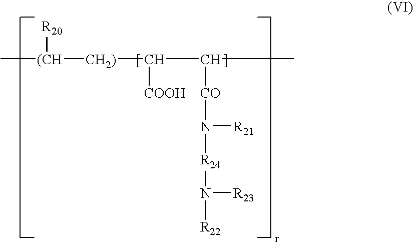

in which R₂₀ represents a hydrogen atom, a CH₃O, CH₃CH₂O or phenyl radical, R₂₁ denotes hydrogen or a lower alkyl radical such as methyl or ethyl, R₂₂ denotes hydrogen or a lower alkyl radical such as methyl or ethyl, R₂₃ denotes a lower alkyl radical such as methyl or ethyl or a radical corresponding to the formula: —R₂₄—N(R₂₂)₂, R₂₄ representing a —CH₂—CH₂—, —CH₂—CH₂—CH₂— or —CH₂—CH(CH₃)— group, R₂₂ having the meanings mentioned above,

as well as the higher homologues of these radicals and containing up to 6 carbon atoms.

(8) amphoteric polymers of the type -D-X-D-X— chosen from:

a) polymers obtained by the action of chloroacetic acid or sodium chloroacetate on compounds comprising at least one unit of formula:

-D-X-D-X-D- (VII)

where D denotes a radical

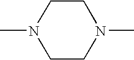

and X denotes the symbol E or E′, E or E′, which may be identical or different, denote a divalent radical which is an alkylene radical with a straight or branched chain containing up to 7 carbon atoms in the main chain, which is unsubstituted or substituted with hydroxyl groups and which can comprise, in addition to the oxygen, nitrogen and sulfur atoms, 1 to 3 aromatic and/or heterocyclic rings; the oxygen, nitrogen and sulfur atoms being present in the form of ether, thioether, sulfoxide, sulfone, sulfonium, alkylamine or alkenylamine groups, hydroxyl, benzylamine, amine oxide, quaternary ammonium, amide, imide, alcohol, ester and/or urethane groups.

b) polymers of formula:

-D-X-D-X— (VII′)

where D denotes a radical

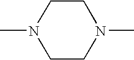

and X denotes the symbol E or E′ and at least once E′; E having the meaning given above and E′ is a divalent radical which is an alkylene radical with a straight or branched chain having up to 7 carbon atoms in the main chain, which is unsubstituted or substituted with one or more hydroxyl radicals and containing one or more nitrogen atoms, the nitrogen atom being substituted with an alkyl chain which is optionally interrupted by an oxygen atom and necessarily comprising one or more carboxyl functions or one or more hydroxyl functions and betainized by reaction with chloroacetic acid or sodium chloroacetate.

(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkanolamine. These copolymers can also comprise other vinyl comonomers such as vinylcaprolactam.

The amphoteric fixing polymers which are particularly preferred according to the invention are those of family (3), such as the copolymers whose CTFA name is octylacrylamide/acrylates/butylaminoethyl methacrylate copolymer, such as the products sold under the names Amphomer, Amhomer LV 71 or Lovocryl 47 by the company National Starch and those of family (4) such as the copolymer of methyl methacrylate/methyl dimethylcarboxymethylammonioethylmethacrylate, for example, sold under the name Diaformer Z301 by the company Sandoz.

The nonionic fixing polymers that can be used according to the present invention are chosen, for example, from:

vinylpyrrolidone homopolymers;

copolymers of vinylpyrrolidone and of vinyl acetate;

polyalkyloxazolines such as the polyethyloxazolines sold by the company Dow Chemical under the names Peox 50 000, Peox 200 000 and Peox 500 000;

vinyl acetate homopolymers, such as the product sold under the name Appretan EM by the company Hoechst, or the product sold under the name Rhodopas A 012 by the company Rhône-Poulenc;

copolymers of vinyl acetate and of acrylic ester; such as the product sold under the name Rhodopas AD 310 by Rhône-Poulenc;

copolymers of vinyl acetate and of ethylene, such as the product sold under the name Appretan TV by the company Hoechst;

copolymers of vinyl acetate and of maleic ester, for example of dibutyl maleate, such as the product sold under the name Appretan MB Extra by the company Hoechst;

copolymers of polyethylene and of maleic anhydride;

alkyl acrylate homopolymers and alkyl methacrylate homopolymers, such as the product sold under the name Micropearl RQ 750 by the company Matsumoto or the product sold under the name Luhydran A 848 S by the company BASF;

acrylic ester copolymers such as, for example, copolymers of alkyl acrylates and of alkyl methacrylates, such as the products sold by the company Rohm & Haas under the names Primal AC-261 K and Eudragit NE 30 D, by the company BASF under the names Acronal 601, Luhydran LR 8833 or 8845, and by the company Hoechst under the names Appretan N 9213 or N 9212;

copolymers of acrylonitrile and of a nonionic monomer chosen, for example, from butadiene and alkyl (meth)acrylates; mention may be made of the products sold under the names Nipol LX 531 B by the company Nippon Zeon or those sold under the name CJ 0601 B by the company Rohm & Haas;

polyurethanes, such as the products sold under the names Acrysol RM 1020 or Acrysol RM 2020 by the company Rohm & Haas, and the products Uraflex XP 401 UZ and Uraflex XP 402 UZ by the company DSM Resins;

copolymers of alkyl acrylate and of urethane, such as the product 8538-33 by the company National Starch;

polyamides, such as the product Estapor LO 11 sold by the company Rhône-Poulenc.

unmodified or chemically modified nonionic guar gums.

The unmodified nonionic guar gums are, for example, the products sold under the name Vidogum GH 175 by the company Unipectine and under the name Jaguar C by the company Meyhall.

The modified nonionic guar gums that can be used according to the invention are preferably modified with C₁-C₆ hydroxyalkyl groups. Mention may be made, for example, of hydroxymethyl, hydroxyethyl, hydroxypropyl and hydroxybutyl groups.

These guar gums are well known in the state of the art and can be prepared, for example, by reacting corresponding alkene oxides, such as, for example, propylene oxides, with guar gum so as to obtain a guar gum modified with hydroxypropyl groups.

Such nonionic guar gums optionally modified with hydroxyalkyl groups are sold, for example, under the trade names Jaguar HP8, Jaguar HP60 and Jaguar HP120, Jaguar DC 293 and Jaguar HP 105 by the company Meyhall or under the name Galactasol 4H4FD2 by the company Aqualon.

The alkyl radicals of the nonionic polymers have from 1 to 6 carbon atoms except where otherwise mentioned.

The nonionic polymers that are most particularly suitable for preparing the compositions in accordance with the invention are those chosen from:

vinyllactam copolymers such as copolymers of vinylpyrrolidone and of vinyl acetate and vinylpyrrolidone/vinyl acetate/vinyl propionate copolymers

the polyvinylcaprolactam Luviskol Plus (BASF)

vinyl acetate homopolymers such as Appretan EM (Hoechst) or Rhodopas A 012 (Rhône-Poulenc)

polyalkyloxazolines such as Peox 50 000 and Peox 500 000 (Dow Chemical)

copolymers of vinyl acetate and of acrylic ester such as Rhodopas AD 310 (Rhône-Poulenc)

copolymers of vinyl acetate and of ethylene, such as Appretan TV (Hoechst)

copolymers of vinyl acetate and of maleic ester, such as Appretan MB Extra (Hoechst)

alkyl acrylate homopolymers and alkyl methacrylate homopolymers, such as Luhydran A 848 S (BASF)

acrylic ester copolymers such as Primal AC-261 K (Rohm & Haas), Acronal 601 (BASF) or Appretan N 9.213 (Hoechst)

copolymers of acrylonitrile and of a nonionic monomer such as CJ 0601 B (Rohm & Haas)

polyurethanes such as Acrysol RM 1020 or Acrysol RM 2020 (Rohm & Haas)

copolymers of alkyl acrylate and of urethane, such as 8538-33 (National Starch)

polyamides such as Estapor LO 11 (Rhône-Poulenc).

According to the invention, it is also possible to use fixing polymers of grafted silicone type comprising a polysiloxane portion and a portion consisting of a nonsilicone organic chain, one of the two portions constituting the main chain of the polymer, the other being grafted onto said main chain. These polymers are described, for example, in patent applications EP-A-0 412 704, EP-A-0 412 707, EP-A-0 640 105 and WO 95/00578, EP-A-0 582 152 and WO 93/23009 and patents U.S. Pat. No. 4,693,935, U.S. Pat. No. 4,728,571 and U.S. Pat. No. 4,972,037. These polymers are preferably anionic or nonionic.

Such polymers are, for example, copolymers that can be obtained by radical polymerization from the monomer mixture consisting of:

a) 50 to 90% by weight of tert-butyl acrylate;

b) 0 to 40% by weight of acrylic acid;

c) 5 to 40% by weight of silicone macromer of formula:

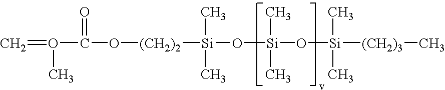

with v being a number from 5 to 700; the weight percentages being calculated relative to the total weight of the monomers.

Other examples of grafted silicone polymers are, in particular, polydimethylsiloxanes (PDMSs) onto which are grafted, via a thiopropylene-type connecting chain, mixed polymer units of the poly(meth)acrylic acid type and of the polyalkyl (meth)acrylate type and polydimethylsiloxanes (PDMSs) onto which are grafted, via a thiopropylene-type connecting chain, polymer units of the polyisobutyl (meth)acrylate type.

Another type of silicone fixing polymer that may be mentioned is the product Luviflex Silk, sold by the company BASF.

Functionalized or nonfunctionalized, silicone or nonsilicone polyurethanes may also be used as fixing polymers.

The polyurethanes particularly intended by the present invention are those disclosed in patents EP 0 751 162, EP 0 637 600, FR 2 743 297 and EP 0 648 485, of which the Applicant is the Proprietor, and patents EP 0 656 021 or WO 94/03510 from the company BASF and EP 0 619 111 from the company National Starch.

As polyurethanes that are particularly suitable for the invention, mention may be made of the products sold under the names Luviset Pur and Luviset Si-Pur by the company BASF.

The relative weight concentration of fixing polymer in the composition is advantageously between 0.05% and 20% by weight and even more advantageously between 0.5% and 10%.

The compositions in accordance with the invention may comprise a conditioner.

A conditioner may advantageously be chosen from the group comprising poly-α-olefins, fluoro oils, plant oils, natural waxes, fluoro waxes, fluoro gums and fatty acid esters, organosiloxanes, amide compounds comprising at least one fatty chain, and ceramides; said agents possibly being present in the form of mixtures.

Among the organosiloxanes used in accordance with the present invention, mention may be made, in a nonlimiting manner, of:

I. Volatile Silicones

These have a boiling point of between 60° C. and 260° C. Among the silicones of this type which are mentioned are:

(i) cyclic silicones of 3 to 7 and preferably 4 to 5 silicon atoms. These are, for example, octamethylcyclotetrasiloxane sold under the name “Volatile Silicone 7207®” by the company Union Carbide or “Silbione 70045 V2®” by the company Rhône-Poulenc, decamethylcyclopentasiloxane sold under the name “Volatile Silicone 7158®” by the company Union Carbide, and “Silbione 70045 V5®” by the company Rhône-Poulenc, and mixtures thereof.

Mention is also made of cyclopolymers of the dimethylsiloxane/methylalkylsiloxane type, such as “Volatile Silicone FZ 3109®” sold by the company Union Carbide, which is a dimethylsiloxane/methyloctylsiloxane cyclocopolymer;

(ii) linear volatile silicones containing 2 to 9 silicon atoms and having a viscosity of less than or equal to 5×10⁻⁶ m²/s at 25° C. An example is hexamethyldisiloxane sold under the name “Silbione 70041 V0.65®” by the company Rhône-Poulenc. This type of product is described in the article by Todd & Byers “Volatile silicone fluids for cosmetics”, Cosmetics and Toiletries, Vol. 91, Jan 76, pages 27-32.

II. Nonvolatile Silicones

These consist mainly of poly(C₁-C₃₅) alkylsiloxanes, polyarylsiloxanes, poly(C₁-C₃₅)alkylarylsiloxanes, silicone gums and resins and organomodified polysiloxanes, as well as mixtures thereof.

Among the polyalkylsiloxanes which may be mentioned mainly are linear polydimethylsiloxanes with a viscosity of greater than 5×10⁻⁶ m²/s, and preferably less than 2.6 m²/s, i.e.:

containing trimethylsilyl end groups, such as, for example, and in a nonlimiting manner, the “Silbione®” oils of the series 70047 sold by the company Rhône-Poulenc, the oil “47 V 500,000®” from Rhône-Poulenc or certain “Viscasil®”s from the company General Electric,

containing trihydroxysilyl end groups, such as the oils of the series “48 V®” from the company Rhône-Poulenc.

In this class of polyalkylsiloxanes, mention may also be made of the polyalkylsiloxanes sold by the company Goldschmidt under the names “Abilwax 9800®” and “Abilwax 9801®”, which are poly(C₁-C₂₀)alkylsiloxanes.

Among the polyalkylarylsiloxanes which may be mentioned are linear and/or branched polydimethylphenylsiloxanes and polydimethyldiphenylsiloxanes, with a viscosity from 10⁻⁵ m²/s to 5×10⁻² m²/s, such as, for example:

the oil “Rhodorsil®” 763 from Rhône-Poulenc,

the “Silbione®” oils of the series 70641 from Rhône-Poulenc, such as the oils “Silbione 70641 V30®” and “Silbione 70641 V200®” from Rhône-Poulenc,

the product “DC 556®” cosmetic grade fluid from Dow Corning,

silicones of the PK series from Bayer, such as “PK20®”,

silicones of the PN and PH series from Bayer, such as “PN 1000®” and “PH 1000®”,

certain oils of the SF series from General Electric, such as “SF 1250®”, “SF 1265®”, “SF 1154®” and “SF 1023®”.

The silicone gums in accordance with the present invention are polydiorganosiloxanes with a high number-average molecular mass of between 200 000 and 1 000 000, which are used alone or as a mixture in a solvent chosen from volatile silicones, polydimethylsiloxane (PDMS) oils, polyphenylmethylsiloxane (PPMS) oils, isoparaffins, methylene chloride, pentane, dodecane, tridecane and tetradecane, or mixtures thereof.

Mention is made, for example, of the compounds having the following structures:

poly[(dimethylsiloxane)/(methylvinylsiloxane)],

poly[(dimethylsiloxane)/(diphenylsiloxane)],

poly[(dimethylsiloxane)/(phenylmethylsiloxane)],

poly[(dimethylsiloxane)/(diphenylsiloxane)/(methylvinylsiloxane)].

Mention may be made, for example, in a nonlimiting manner, of the following mixtures:

1) mixtures formed from a polydimethylsiloxane which is hydroxylated at the end of the chain (Dimethiconol according to the CTFA nomenclature) and of a cyclic polydimethylsiloxane (Cyclomethicone according to the CTFA nomenclature), such as the product “Q2 1401®” sold by the company Dow Corning;

2) mixtures formed from a polydimethylsiloxane gum with a cyclic silicone, such as the product “SF 1214 Silicone Fluid®” from General Electric, which is an “SE 30®” gum of MW 500 000 (\overline{Mn}) dissolved in “SF 1202 Silicone Fluid®” (decamethylcyclopentasiloxane);

3) mixtures of two PDMSs of different viscosities, in particular of a PDMS gum and of a PDMS oil, such as the products “SF 1236®” and “CF 1241®” from the company General Electric. The product “SF 1236®” is the mixture of an “SE 30®” gum defined above with a viscosity of 20 m²/s and of an “SF 96®” oil with a viscosity of 5×10⁻⁶ m²/s (15% “SE 30®” gum and 85% “SF 96®” oil).

The product “CF 1241®” is the mixture of an “SE 30®” gum (33%) and of a PDMS (67%) with a viscosity of 10⁻³ m²/s.

The polyorganosiloxane resins which can be used in accordance with the invention are crosslinked siloxane systems containing the units: R₂SiO_{2/2}, RSiO_{3/2} and SiO_{4/2} in which R represents a hydrocarbon-based group containing 1 to 6 carbon atoms or a phenyl group. Among these products, those particularly preferred are the ones in which R denotes a lower alkyl radical or a phenyl radical.

Among these resins, mention may be made of the product sold under the name “Dow Corning 593®” or those sold under the names “Silicone Fluid SS 4230” and “Silicone Fluid SS 4267” by the company General Electric and which are dimethyl/trimethylpolysiloxanes.

The organomodified silicones in accordance with the present invention are silicones as defined above, whose general structure comprises one or more organofunctional groups directly attached to the siloxane chain or attached via a hydrocarbon-based radical.

Mention is made, for example, of silicones comprising:

a) perfluoro groups such as trifluoroalkyl groups, such as, for example, those sold by the company General Electric under the names “FF.150 Fluorosilicone Fluid®” or by the company Shin-Etsu under the names “X-22-819®”, “X-22-82®”, “X-22-821®” and “X-22-822®”;

b) hydroxyacylamino groups such as, for example, those described in patent application EP-A-0 342 834 and in particular the silicone sold by the company Dow Corning under the name “Q2-8413®”;

c) thiol groups such as the silicones “X 2-8360®” from the company Dow Corning or “GP 72A®” and “GP 71®” from Genesee;

d) substituted or unsubstituted amine groups, such as in “GP 4 Silicone Fluid®” from Genesee, “GP 7100®”/from Genesee, “Q2 8220®” from Dow Corning, “AFL 40®” from Union Carbide or the silicone known as “Amodimethicone” in the CTFA dictionary;

e) carboxylate groups, such as the products described in patent EP 186 507 from Chisso Corporation;

f) hydroxylated groups, such as the polyorganosiloxanes containing a hydroxyalkyl function which are described in patent application FR-85/16334, corresponding to the following formula:

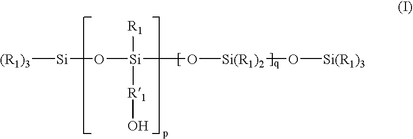

in which:

the radicals R₁, which may be identical or different, are chosen from methyl and phenyl radicals, at least 60 mol % of the radicals R₁ being methyl;

the radical R′₁ is a C₂-C₁₈ hydrocarbon-based divalent alkylene chain unit;

p is between 1 and 30 inclusive;

q is between 1 and 150 inclusive.

Mention may be made most particularly of the product sold by Dow Corning under the name DC 190;

g) alkoxylated groups, such as in the silicone copolymer “F 755®” from SWS Silicones and the products “Abilwax 2428®”, “Abilwax 2434®” and “Abilwax 2440®” from the company Goldschmidt;

h) acyloxyalkyl groups such as, for example, the polyorganopolysiloxanes described in patent application FR-88/17433, corresponding to the following formula:

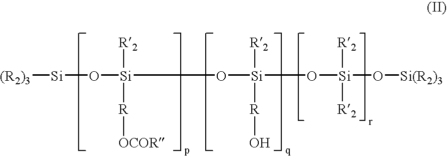

in which:

R₂ denotes methyl, phenyl, OCOR″ or hydroxyl; only one of the radicals R₂ per silicon atom can be OH;

R₁₂ denotes methyl or phenyl; at least 60 mol % of all of the radicals R₂ and R₁₂ are methyl;

R″ denotes C₈-C₂₀ alkyl or alkenyl;

R denotes a linear or branched C₂-C₁₈ divalent hydrocarbon-based alkylene;

r is between 1 and 120 inclusive;

p is between 1 and 30 inclusive;

q is 0 or is less than 0.5 p, p+q being between 1 and 30 inclusive;

the polyorganosiloxanes of formula (II) can contain groups

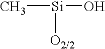

in proportions not exceeding 15% of the sum p+q+r;

i) quaternary ammonium groups, such as in the products “X2 81 08” and “X2 81 09®” and the product “ABIL K 3270®” from the company Goldschmidt;

j) amphoteric or betaine groups, such as in the product sold by the company Goldschmidt under the name “Abil B 9950®”;

k) bisulfite groups, such as in the products sold by the company Goldschmidt under the names “Abil S 201®” and “Abil S 255®”;

l) oxyalkylenated groups.

The viscosities of the silicones can be determined in particular by ASTM standard D445-97 (viscometry).

When the conditioning agent in the composition according to the invention is a hydrocarbon, it is a linear or branched C₈-C₃₀₀ hydrocarbon. Among the hydrocarbons, that are liquid at room temperature, corresponding to this definition, mention may be made in particular of isododecane, isohexadecane and its isomers (such as 2,2,4,4,6,6-heptamethylnonane), isoeicosane, isotetracosane and isomers of said compounds. Isododecane or an isomer thereof is preferably used according to the invention.

When the conditioning agent is a fatty alcohol, it is of the linear or branched, saturated or unsaturated, C₈-C₃₀ type, and among those mention may be made of 2-butyloctanol, lauryl alcohol, oleyl alcohol, isocetyl alcohol, isostearyl alcohol and behenyl alcohol.

When the conditioning agent is a fatty ester, it may be either an ester of a C₈-C₃₀ fatty acid and of a C₁-C₃₀ alcohol or an ester of a C₁-C₇ acid or diacid and of a C₈-C₃₀ fatty alcohol. Among these esters, mention may be made of ethyl, isopropyl, 2-ethylhexyl and 2-octyldecyl palmitate, isopropyl, butyl, cetyl and 2-octyldecyl myristate, butyl and hexyl stearate, hexyl and 2-hexyldecyl laurate, isononyl isononanoate and dioctyl malate.

The ceramides or analogues, such as the glycoceramides which can be used in the compositions according to the invention, are known per se and are natural or synthetic molecules which can correspond to the following general formula:

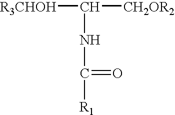

in which:

R₁ denotes a linear or branched, saturated or unsaturated alkyl radical derived from C₁₄-C₃₀ fatty acids, it being possible for this radical to be substituted with a hydroxyl group in the α position, or a hydroxyl group in the ω position esterified with a saturated or unsaturated C₁₆-C₃₀ fatty acid;

R₂ denotes a hydrogen atom or a (glycosyl)ₙ, (galactosyl)ₘ or sulfogalactosyl radical, in which n is an integer ranging from 1 to 4 and m is an integer ranging from 1 to 8.

R₃ denotes a saturated or unsaturated C₁₅-C₂₆ hydrocarbon-based radical in the α position, it being possible for this radical to be substituted with one or more C₁-C₁₄ alkyl radicals;

it being understood that, in the case of the natural ceramides or glycoceramides, R₃ can also denote a C₁₅-C₂₆ α-hydroxyalkyl radical, the hydroxyl group optionally being esterified with a C₁₆-C₃₀ α-hydroxy acid.

In the context of the present invention, the preferred ceramides are those described by Downing in Arch. Dermatol., Vol. 123, 1381-1384, 1987, or those described in French patent FR-2 673 179, the structures of which may be as follows: [lacuna]

The ceramides more particularly preferred according to the invention are the compounds for which R₁ denotes a saturated or unsaturated alkyl derived from C₁₆-C₂₂ fatty acids; R₂ denotes a hydrogen atom; and R₃ denotes a saturated linear C₁₅ radical.

Such compounds are, for example:

N-linoleoyldihydrosphingosine,

N-oleoyldihydrosphingosine,

N-palmitoyldihydrosphingosine,

N-stearoyldihydrosphingosine,

N-behenoyldihydrosphingosine,

or mixtures of these compounds.

Even more preferably, the ceramides for which R₁ denotes a saturated or unsaturated alkyl radical derived from fatty acids; R₂ denotes a galactosyl or sulfogalactosyl radical; and R₃ denotes a —CH═CH—(CH₂)₁₂—CH₃ group, are used.

By way of example, mention may be made of the product consisting of a mixture of these compounds, sold under the trade name Glycocer by the company Waitaki International Biosciences.

The cosmetically acceptable medium preferably consists of water or one or more cosmetically acceptable solvents such as alcohols or water/solvent(s) mixtures, the solvents preferably being C₁-C₄ alcohols.

Among these alcohols that may be mentioned are ethanol and isopropanol. Ethanol is particularly preferred.

Preferably, the concentration of organic solvent(s) is less than 30% and even more preferably less than 20%.

The composition of the invention may also contain at least one additive chosen from nonionic, anionic, cationic, amphoteric or zwitterionic surfactants, fragrances, screening agents, preserving agents, proteins, vitamins, nonionic, anionic, cationic, amphoteric or zwitterionic polymers other than those of the invention, mineral, plant or synthetic oils, thickeners and any other additive conventionally used in cosmetic compositions, antidandruff agents, agents for preventing hair loss, electrolytes, colorants, pigments, moisturizers such as glycerol and other polyols, and reducing agents.

These additives are optionally present in the composition according to the invention in proportions that may advantageously range from 0.001% to 20% by weight relative to the total weight of the composition. The precise amount of each additive depends on its nature and may readily be determined by a person skilled in the art, and will depend on the selected hair application.

Needless to say, a person skilled in the art will take care to select the optional compound(s) to be added to the composition according to the invention such that the advantageous properties intrinsically associated with the composition in accordance with the invention are not, or are not substantially, adversely affected by the envisaged addition.

The compositions of the invention may be used for manufacturing numerous hair products such as, for example, products for fixing and/or holding the hair, conditioning products, for instance sheen formulations, or haircare products.

These hair formulations according to the invention will be packaged in aerosol form or in a pump-bottle, preferably without a propellant.

The invention may be understood more clearly with the aid of the nonlimiting examples that follow, which constitute advantageous embodiments of the compositions in accordance with the invention.

All the amounts are expressed as relative percentages by weight relative to the total weight of the composition, and a.m. means active material.

### EXAMPLES:

The compositions below in accordance with the invention are prepared. Formulations A and B are aqueous compositions, and formulations C and D are aqueous-alcoholic compositions with high ethanol contents. Compositions E, F and G contain, besides a thickening polymer with an aminoplast-ether skeleton, either a conditioner or a fixing polymer.

Mousse in Aqueous Solvent With Propellant:

| Formulation A | |
|---|---|
| Pure Thix TX 1442⁽¹⁾ | 0.1% |
| Water qs | 89.9% |
| Butane 3.2 | 10% |

⁽¹⁾Polymer of tetrakis methoxy methyl

glycoluril/hydrophobic modified polyether structure sold by Sud Chemie

Mousse in Aqueous Solvent Without Propellant:

The following composition is packaged in a Foam pump device reference F2 sold by Airspray.

| Formulation B | |
|---|---|
| Pure Thix TX 1442 | 0.3% |
| Water qs | 100% |

The mousses obtained from formulations A and B are pleasant, and have respective densities of 0.10 and 0.19 g/cm³, the density being measured according to the method described in patent FR 2 704 771, of which the Applicant is the proprietor.

Mousse in Aqueous-Alcoholic Solvent With Propellant:

| Formulation C | |
|---|---|
| Pure Thix TX 1442 | 1% |
| Ethanol | 30% |
| Water qs | 89% |
| Propellant (Butane 3.2) | 10% |

Mousse in Aqueous-Alcoholic Solvent Without Propellant:

The Foam pump device reference F2 from Airspray is again used.

| Formulation D | |
|---|---|
| Pure Thix TX 1442 | 1% |
| Ethanol | 30% |
| Water qs | 100% |

The mousses obtained from formulations C and D are pleasant, despite their high alcohol content, and have respective densities of 0.035 and 0.12 g/cm³, the density being measured according to the method described in patent FR 2 704 771, of which the Applicant is the proprietor.

Other compositions are prepared, this time comprising, besides a polymer in accordance with the invention, a fixing polymer or a conditioner.

Mousse With Anionic Fixing Polymer:

| Formulation E | |
|---|---|
| Pure Thix TX 1442 | 0.5% |
| Acrylidone LM sold by ISP | 0.5% |
| Aminomethylpropanol qs neutralization | to 100% |
| Water qs | 100% |

Mousses With Conditioners:

| Formulation F | |
|---|---|
| Pure Thix TX 1442 | 1% |
| Luviquat FC 370 sold by BASF | 2.5% |
| Water qs | 100% |

| Formulation G | |
|---|---|
| Pure Thix TX 1442 | 1% |
| DC 939 Emulsion sold by Dow Corning | 1.43% |
| Water qs | 100% |

Formulations E, F and G give good-quality mousses, despite the small amount of foaming agent, and have a density in the region of 0.1 g/cm³, the density being measured according to the method described in patent FR 2 704 771, of which the Applicant is the proprietor.

1. A cosmetic hair composition in mousse form, comprising, in a cosmetically acceptable medium, at least one thickening polymer with an aminoplast-ether skeleton.

2. The composition as claimed in claim 1, characterized in that the aminoplast-ether polymer(s) is (are) chosen from those containing at least one unit of structure (I) below: 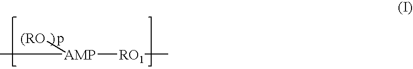
which:
AMP is an aminoplast residue with alkylene units,
R denotes a hydrogen atom, a C₁-C₄ alkyl radical or a C₁-C₄ acyl radical,
RO₁ is a divalent alkyleneoxy residue,
p denotes a positive integer,
the group(s) OR being linked to the alkylene units of the AMP residue.

3. The composition as claimed in claim 1 or 2, characterized in that the polymer(s) with an aminoplast-ether skeleton is (are) chosen from those containing at least one unit of structure (II) below: 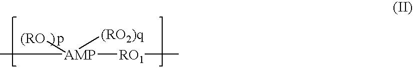
in which:
AMP, R, RO₁ and p have the same meaning as in claim 2,
RO₂ is a hydrophobic group other than RO linked to AMP via a hetero atom and comprising at least two carbon atoms, and
q is a positive integer.

4. The composition as claimed in claimed 3, characterized in that the aminoplast-ether polymer(s) is (are) chosen from the polymers of structure (III) or (IIIa) below: 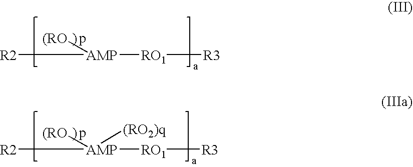
in which:
AMP, R, RO₁, RO₂, p and q have the same meaning as in claims 2 and 3,
R2 or R3, which may be identical or different, represent an end group that can denote a hydrogen atom, a group RO₁H, a group RO₂H, a group AMP(OR)ₚ or any monofunctional group such as alkyl, cycloalkyl, aryl, aralkyl, alkylaryl, alkyloxyalkyl, aryloxyalkyl or cycloalkoxyalkyl,
a being a number greater than 1 and preferably greater than 2.

5. The composition as claimed in any one of claims 2 to 4, characterized in that the aminoplast residues bearing the groups OR thereof are chosen from those of structures (IV) to (XV) below: 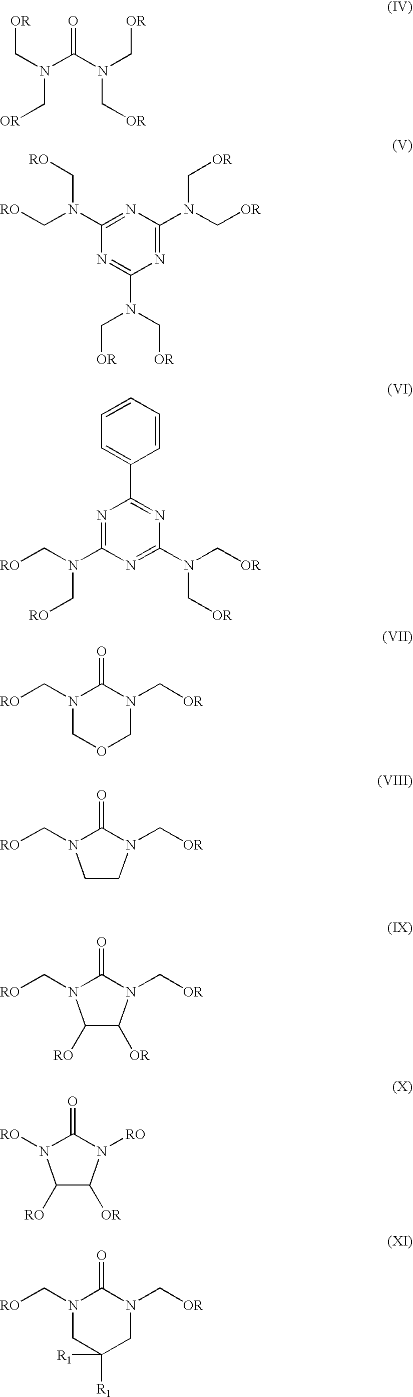 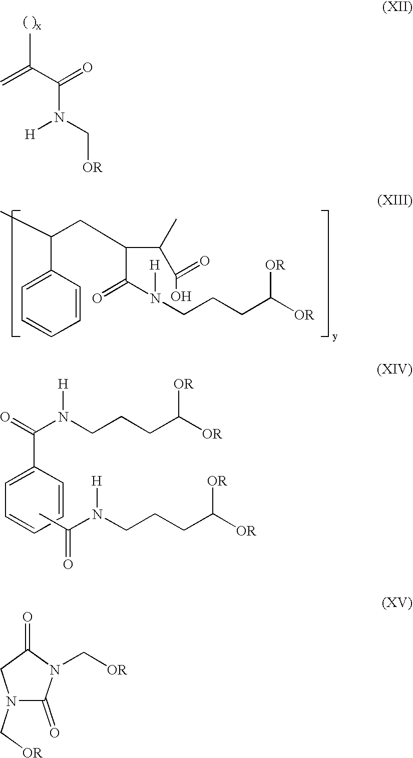
R having the same meaning as in claim 2,
R1 denotes C₁-C₄ alkyl,
y is a number at least equal to 2,
x denotes 0 or 1.

6. The composition as claimed in any one of claims 2 to 4, characterized in that the aminoplast residues bearing the groups OR thereof are chosen from those of structure (XVI) below: 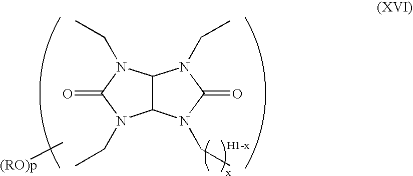
in which R, p and x have the same meanings as in claims 2 and 5.

7. The composition as claimed in any one of claims 2 to 6, characterized in that the alkyleneoxy residues are those corresponding to the diols of general formula (XVII) below:
HO-(ZO)y-(Z1(Z2O)w)t-(Z′O)y′-Z3OH (XVII),
y and y′ being numbers ranging from 0 to 1000,
t and w being numbers ranging from 0 to 10,
Z, Z′, Z2 and Z3 are C₂-C₄ alkylene radicals and preferably radicals —CH2-CH(Z4)- and —CH2-CH(Z4)-CH2-,
Z1 being a linear or cyclic, branched or unbranched, aromatic or nonaromatic radical optionally comprising one or more hetero atoms and containing from 1 to 40 carbon atoms,
Z4 denoting a hydrogen atom or a C₁-C₄ alkyl radical or a C₁-C₃ acyl radical, it being understood that at least one of the radicals Z4 of the radicals Z, Z′, Z2 and Z3 is other than acyl.

8. The composition as claimed in claim 7, characterized in that the radical Z4 denotes a hydrogen atom or a methyl radical.

9. The composition as claimed in claims 7 and 8, characterized in that t=0 and in that Z, Z′and Z3 denote —CH2CH2-, at least one from among y and y′ being other than 0.

10. The composition as claimed in any one of the preceding claims, characterized in that the polymers with an aminoplast-ether skeleton are chosen from the following products:
PEG-180/Octoxynol-40/TMMG Copolymer,
PEG-180/Laureth-50/TMMG Copolymer,
Polyether-1
Tetrakis methoxy methyl glycoluril/hydrophobic modified polyether.

11. The composition as claimed in any one of claims 1 to 10, characterized in that the polymers with an aminoplast-ether skeleton are used in an amount that can range from 0.05% to 3% by weight relative to the total weight of the composition.

12. The composition as claimed in any one of claims 1 to 11, characterized in that the polymers with an aminoplast-ether skeleton are used in an amount that can range from 0.3% to 2% by weight relative to the total weight of the composition.

13. The composition as claimed in any one of the preceding claims, characterized in that it also comprises at least one fixing polymer.

14. The composition as claimed in claim 12, characterized in that the relative weight concentration of fixing polymer(s) is between 0.05% and 20% by weight and preferably between 0.5% and 10%.

15. The composition as claimed in claims 13 and 14, characterized in that the fixing polymer is an anionic polymer chosen from:
polymers comprising carboxylic units derived from unsaturated monocarboxylic or dicarboxylic acid monomers of formula: 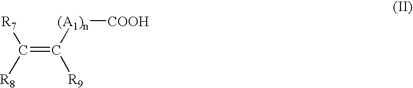
in which n is an integer from 0 to 10, A denotes a methylene group, optionally connected to the carbon atom of the unsaturated group, or to the neighboring methylene group when n is greater than 1, via a hetero atom such as oxygen or sulfur, R₇ denotes a hydrogen atom or a phenyl or benzyl group, R₈ denotes a hydrogen atom or a lower alkyl or carboxyl group, R₉ denotes a hydrogen atom, a lower alkyl group or a —CH₂—COOH, phenyl or benzyl group;
polymers comprising units derived from sulfonic acid, such as vinylsulfonic, styrenesulfonic or acrylamidoalkylsulfonic units.

16. The composition as claimed in claims 13 and 14, characterized in that the fixing polymer is an amphoteric polymer chosen from polymers comprising units derived from:
a) at least one monomer chosen from acrylamides and methacrylamides substituted on the nitrogen with an alkyl radical,
b) at least one acidic comonomer containing one or more reactive carboxylic groups, and
c) at least one basic comonomer such as esters containing primary, secondary, tertiary and quaternary amine substituents of acrylic and methacrylic acids and the product of quaternization of dimethylaminoethyl methacrylate with dimethyl or diethyl sulfate.

17. The composition as claimed in claims 13 and 14, characterized in that the fixing polymer is a nonionic polymer chosen from:
polyalkyloxazolines;
vinyl acetate homopolymers;
copolymers of vinyl acetate and of acrylic ester;
copolymers of vinyl acetate and of ethylene;
copolymers of vinyl acetate and of maleic ester;
copolymers of polyethylene and of maleic anhydride;
alkyl acrylate homopolymers and alkyl methacrylate homopolymers;
acrylic ester copolymers such as, for example, copolymers of alkyl acrylates and of alkyl methacrylates;
copolymers of acrylonitrile and of a nonionic monomer chosen, for example, from butadiene and alkyl (meth)acrylates;
copolymers of alkyl acrylate and of urethane;
polyamides; and
unmodified or chemically modified nonionic guar gums.

18. The composition as claimed in claims 13 and 14, characterized in that the fixing polymer is a cationic polymer chosen from:
the copolymer of acrylamide and of dimethylaminoethyl methacrylate quaternized with dimethyl sulfate,
copolymers of acrylamide and of methacryloyloxyethyltrimethylammonium chloride,
the copolymer of acrylamide and of methacryloyloxyethyltrimethylammonium methosulfate,
quaternized or nonquaternized vinylpyrrolidone/dialkylaminoalkyl acrylate or methacrylate copolymers,
dimethylaminoethyl methacrylate/vinylcaprolactam/vinylpyrrolidone terpolymers,
the quaternized vinylpyrrolidone/dimethyl-aminopropylmethacrylamide copolymer,
quaternized polysaccharides, such as guar gums containing trialkylammonium cationic groups;
quaternary copolymers of vinylpyrrolidone and of vinylimidazole,
chitosans or salts thereof,
cationic cellulose derivatives.

19. The composition as claimed in claims 13 and 14, characterized in that the fixing polymer is a functionalized or unfunctionalized, silicone or nonsilicone polyurethane.

20. The composition as claimed in claims 13 and 14, characterized in that the fixing polymer is a polymer of grafted silicone type comprising a polysiloxane portion and a portion consisting of a nonsilicone organic chain, one of the two portions constituting the main chain of the polymer, the other being grafted onto said main chain.

21. The composition as claimed in any one of the preceding claims, characterized in that it also comprises a conditioner from the group comprising poly-α-olefins, fluoro oils, plant oils, natural waxes, fluoro waxes, fluoro gums and fatty acid esters, organosiloxanes, amide compounds comprising at least one fatty chain, and ceramides; said agents possibly being present in the form of mixtures.

22. The composition as claimed in any one of the preceding claims, characterized in that it contains at least one additive chosen from nonionic, anionic, cationic, amphoteric or zwitterionic surfactants, fragrances, screening agents, preserving agents, proteins, vitamins, nonionic, anionic, cationic, amphoteric or zwitterionic polymers other than those of the invention, mineral, plant or synthetic oils, thickeners and any other additive conventionally used in cosmetic compositions, antidandruff agents, agents for preventing hair loss, electrolytes, colorants, pigments, moisturizers such as glycerol and other polyols, and reducing agents.

23. A cosmetic hair process, characterized in that it consists in applying to the hair a composition in accordance with any one of the preceding claims.

24. The use of a polymer with an aminoplast-ether skeleton, as a foaming agent.
